(19) United States
(12) Patent Application Publication
(10) Pub. No.: US 2004/0172056 A1
(43) Pub. Date: Sep. 2, 2004

(54) Bifurcated aneurysm buttress arrangement

(76) Inventors: Lee R. Guterman, Amherst, NY (US); Paul A. LaDuca, Buffalo, NY (US); Robert C. LaDuca, Santa Cruz, CA (US)

Correspondence Address: Donald N. Halgren, 35 Central Street, Manchester, MA, 01944, US

(21) Appl. No.: 10/715,875

(22) Filed: Nov. 18, 2003

Related U.S. Application Data
(63) Continuation-in-part of application No. 10/618,841, filed on Jul. 14, 2003.
(60) Provisional application No. 60/395,974, filed on Jul. 12, 2002.

(51) Int. Cl. A61F 2/06

(52) U.S. Cl. 606/200; 623/1.11; 623/1.15

(57) ABSTRACT

An aneurysm buttressing arrangement for covering an aneurysm opening in an intracranial aneurysm, for temporary placement thereadjacent, to prevent escape of embolitic agents from that aneurysm. The buttressing arrangement comprises an elongated delivery wire having a proximal end and a distal end. An expandable scaffold of wire mesh is arranged on the distal end of the delivery wire. The scaffold comprises a generally “U” shaped arrangement of wire mesh attached to the delivery wire.

### BACKGROUND OF THE INVENTION

1. Field of the Invention

This invention relates to methodology and apparatus for the treatment of an aneurysm particularly intracranial aneurysms disposed at a bifurcated vessel, and is a continuation in part application of our co-pending U.S. patent application Ser. No. 10/618,841, filed 14 Jul. 2003, which is based upon provisional application serial No. 60/395,974, filed 12 Jul. 2002, each of which are incorporated herein by reference, in their entirety.

2. Prior Art

The present invention relates to the treatment of vascular aneurysms and in particular to methods and devices for filling aneurysms located at the juncture of a bifurcation of a vessel, while maintaining patency of the vessel arrangement.

Various implantable medical devices have been developed for treating ailments in the vascular system. Vaso-occlusive devices have been used extensively in closing regions of the vascular system. These devices are especially useful in treating aneurysms. Vascular aneurysms are formed as a result of a weakening in the wall of an artery and subsequent ballooning of the artery wall. Aneurysms are often a site of internal bleeding and, catastrophically, result in hemorrhagic strokes. A variety of different embolic agents are known to be suitable for treatment of such aneurysms. These treatments are commonly known as “artificial vaso-occlusion.”

Recent advancements in the artificial occlusion of vessels and aneurysms have occurred mostly due to the improvements in delivery and implantation of metal coils as vaso-occlusive devices. Implantable metal coils that are useful in artificial occlusion devices in vasculature lumens or aneurysms are herein referred to as “vaso-occlusive coils”.

Vaso-occlusive coils are generally constructed of wire, usually made of a metal or metal alloy, that is first wound into a shape such as a sphere or helix. Many such devices are introduced to the selected target site through a catheter in a stretched linear form. The vaso-occlusive device may assume an irregular shape upon discharge of the device from the distal end of the catheter. A variety of vaso-occlusive coils and braids are known. For instance, U.S. Pat. No. 4,994,069, to Ritchart et al., shows a flexible, preferably coiled, wire for use in small vessel vaso-occlusion. These coils are described as being between 0.010 and 0.030 inches in diameter. The wire used to make up the coils may be, for instance, 0.002 to 0.006 in ches in diameter. Tungsten, platinum, and gold threads or wires are said to be preferred. These devices may be used to fill aneurysms.

It is common for these vaso-occlusive devices to be delivered through microcatheters such as the type shown in U.S. Pat. No. 4,739,768, to Engelson. These microcatheters track a guidewire to a point just proximal or within the desired occlusion site. The vaso-occlusive coils are then advanced through the microcatheter, once the guidewire is removed, and out the distal end hole so to at least partially fill the selected site and create occlusion within the aneurysm. Experiments have indicated that, at most, 40% of the aneurysm is filled with coils. The remainder is filled with naturally occurring thrombus.

In addition to the various types of space-filling mechanisms and geometries of vaso-occlusive coils, other particularized features of coil designs, such as mechanisms for delivering vaso-occlusive coils through delivery catheters and implanting them in desired occlusion sites, have also been described. Examples of such vaso-occlusive devices based upon their delivery mechanisms include pushable coils (Ritchart et al., discussed above), mechanically detachable vaso-occlusive devices (U.S. Pat. No. 5,261,916 to Engelson or U.S. Pat. No. 5,250,071 to Palermo), or electrolytically detachable vaso-occlusive devices (U.S. Pat. Nos. 5,122,136 and 5,354,295 to Guglielmi et al.). Other prior art such as U.S. Pat. No. 5,916,235 to Guglielmi discloses methods and apparatus which have similar characteristics and limitations for not facilitating buttressing, delivery or tracking or the like.

However, after, or perhaps during delivery of such a coil into the aneurysm, there is a risk that a portion of the coil might migrate out of the aneurysm entrance zone and into the feeding vessel. This is especially true in aneurysms where the diameter of the aneurysm neck approaches the diameter of the aneurysm body in a 1:1 ratio. The presence of such a coil in that feeding vessel may cause the undesirable response of causing an occlusion there. Also, there is a quantifiable risk that the blood flow in the vessel and the aneurysm may induce movement of the coil farther out of the aneurysm, resulting in a more thoroughly developed embolus in the patent vessel. Being that coils are constructed from very low gauge wire, the coil mass can compact resulting in aneurysm re-canalization.

Furthermore, one type of aneurysm, commonly known as a “wide-neck aneurysm” is known to present particular difficulty in the placement and retention of vaso-occlusive coils. Wide-neck aneurysms are herein referred to as aneurysms of vessel walls having a neck or an “entrance zone” from the adjacent vessel, which entrance zone has a diameter of either (1) at least 80% of the largest diameter of the aneurysm; or (2) is clinically observed to be too wide effectively to retain vaso-occlusive coils that are deployed using the techniques discussed herein.

Vaso-occlusive coils lacking substantial secondary shape strength may also be difficult to maintain in position within an aneurysm no matter how skillfully they are placed. This may also be true of coils that have a secondary shape. For example, a 3D coil that takes a spherical shape may be herniated out of the aneurysm into the parent vessel if the neck is too wide. Using the buttressing device of the present invention permits the coils to be held in the aneurysm until a critical mass of coils is achieved within the aneurysm so that the coil mass will not move when the buttressing device is withdrawn.

A few devices have been disclosed for maintaining the presence of vaso-occlusive coils within an aneurysm. One such device is a retainer for retaining coils within the aneurysm cavity. The retainer device is released into the vessel exterior to the aneurysm. The device is held in place via the presence of radial pressure on the vessel wall. After the device is released and set in an appropriate place, a microcatheter is inserted into the lumen so that the distal end of the catheter is inserted into the aneurysm cavity. One or more vaso-occlusive devices is then introduced into the aneurysm cavity. The retainer device maintains the presence of the vaso-occlusive devices within the aneurysm whether it is a large-mouth aneurysm or not.

Another approach to filling intracranial aneurysms includes the use of injectable fluids or suspensions, such as microfibrillar collagen, various polymeric beads, and polyvinyl alcohol foal. These polymeric agents may additionally be crosslinked, sometimes in vivo to extend the persistence of the agent at the vascular site. These agents may be introduced into the vasculature through any of a variety of known catheters. After introduction, the deployed materials form a solid space-filling mass. Other materials, including polymeric resins, typically cyanoacrylates, hydrogels and other gels, fibrin glues, and calcium binding seaweed extracts are also employed as injectable vaso-occlusive materials. These materials may be mixed with a radio-opaque contrast material or made radio-opaque by the addition of a tantalum powder.

The delivery of liquid embolic agents into aneurysms in general has numerous obstacles. The viscosity of the material makes delivery difficult, and leads to run on even after the pressure head has been removed from the delivery catheter. Inadequate opacification of the material makes it difficult to see. As a result it can leak into the parent vessel. This can result in vessel occlusion and distal embolization into the organ's vascular bed. To date, these materials have been delivered using an inflated balloon adjacent to the abnormality to be treated. Inflation of the balloon during delivery leads to temporary vessel occlusion and can result in downstream organ ischemia and even infarction.

Thus, notwithstanding the various efforts in the prior art, there remains a need for an embolic deployment system which enables the filling and sealing of an aneurysm particularly in a bifurcated vessel while minimizing the risk of leakage and subsequent migration of any material delivered into the aneurysm, and enabling perfusion during the deployment process.

There also is a need for an aneurysm buttress arrangement which permits blood flow out of the aneurysm, particularly at a bifurcated site, while the aneurysm is being filled with embolic material so that the intra aneurysm pressure does not decrease. There is further need for a buttress arrangement suited for bifurcated sites, which buttress permits flow between adjacent vessels around and/or through the buttress device.

Thus it is an object of the present invention to overcome the insufficiencies of the prior art.

### BRIEF SUMMARY OF THE INVENTION

The present invention relates to a method of filling and buttressing an intracranial aneurysm such as may be located at the juncture of a bifurcated vessel arrangement. The method includes the steps of transluminally positioning a buttress scaffold across or in an opening of an aneurysm at the location of a bifurcated vessel so as to block off and isolate that aneurysm cavity in a sidewall of that bifurcated vessel. Media such as embolitic agents, coils, and/or polymers may then be introduced into that cavity within the sidewall of the bifurcated vessel. The cavity is often of a bulbous shape having a neck portion of no greater than about one-half of the diameter of the bulbous portion, although some aneurysms may have a neck portion as wide as the body of the aneurysm itself.

The bulbous scaffold is arranged on the distal end of an elongated delivery wire or push wire much like a guide wire. The scaffolding itself may be preferably comprised of a mesh or braid of wire, comprised of a memory metal or polymeric fibers and/or a plastic, or a co-weave combination thereof shaped “generally” as a prolate spheroid and disposed in the target vessel in the manner of a generally U-shaped loop. A further example of one preferred embodiment of the present invention may be the scaffold being comprised of a shaped inflatable and deflatable balloon, which balloon may preferably shapably conform to the inventive loop shape. The loop shape of the present invention is defined by two proximal ends which taper into a thinner diameter from a larger diameter at the distalmost end of the loop. The loop both at its distalmost end and at its proximalmost ends are preferably circular in cross section, but may be oval or somewhat rectangular. The loop is expandable and defines a variable central opening-along its innermost periphery thereof.

The buttress scaffold loop in one preferred embodiment may be comprised of an array of flexible interwoven nitinol wires, stainless steel wires or filaments of polymer. A typical pic per inch count for the braided mesh ranges from a low of about 1-20 ppi to a high of about 1000 ppi based on the radial expansion required for a given target vessel aneurysm.

Target vessel sizes may range from about 1 mm to about 6 mm. The individual carrier wire used in a braided mesh loop may have a diameter ranging from about 0.00075 inch to about 0.003 inches (1 micron to 1 cm.). The carrier-counts for the braided mesh may range from about 20 to about 48 carriers, with the denser braid configurations designed to permit enhanced resistance to embolic material leaking beyond the aneurysm neck into the parent vessel while the scaffold device is in place. Denser braided mesh will also provide a greater outwardly directed force than less dense braided mesh, and will therefore hold the deposited embolic coils in place in the aneurysm. Typical braided mesh construction is either a one over, one under, or a two over, two under filament crossing pattern. The braided mesh loop may be formed by heat setting, mechanical bending or other means so as to take on a set expanded shape. The braided mesh may also be electrically polished, and/or chemically etched along a partial section or its entirety. Surface markers such as for example, mechanically attached bands, rings or adhered coatings to increase the fluoroscopic visibility of the scaffold device or at distinct marker points along the loop may also be applied. Hydrophilic coatings, friction reducing coatings or any coating or medicament which may speed healing or enhance the surface properties of an operation associated with the insertion of such a braided mesh loop may be utilized therewith.

The control shaft in one preferred embodiment may be solid such as a stainless steel wire sized appropriately to the overall device size. Such a control shaft may be Teflon coated either partially or along its entire length, with a taper from about 0.12 to 0.03 inches at its distal end. The braided loop of the present invention is intended to act as an atraumatic means of supporting the scaffold device against the main vessel branch of the bifurcation.

The wire mesh loop is preferably delivered through a micro delivery catheter pushed through the main vessel and into the bifurcated target area. The push wire proximal of the mesh loop may be advanced so as to only extend the wire mesh loop only a portion of the way out of the distal end of the micro delivery catheter. This will permit one preferred means of control for the ultimate diameter of the opened distalmost wire mesh loop, without having to move two control wires relative to one another.

The wire mesh loop in its “delivery” configuration in a distal end of a micro catheter is enclosed in a fully collapsed orientation. The inside diameter of a typical micro delivery catheter in which such a wire mesh loop would be loaded and tracked through the vasculature of a patient may range in diameter from about 0.010 inch to about 0.1 inch. The wire mesh loop scaffold is advanced and removed from the aneurysm site via such micro catheter. The collapse of a loop of braided mesh upon retrieval and/or removal of such a wire mesh loop device is a result of the braided mesh loop passing over and being compressed by the annular distalmost lip of the micro delivery catheter. The shape of the distalmost end of the loop is generally spherical or elliptical, in a transition from a teardrop shape to a sphere, it is constructed so as to conform to the local target anatomy if the density of the distalmost mesh of the loop is also selected so as to conform to that target anatomy.

A typical aneurysm “sac” is often located at the locus of intersection of a first and a second blood vessel or channel which is fed by a main vessel channel. The aneurysm sac at such a bifurcation typically has a neck portion which is approximately one-half of the diameter of the enlarged or bulbous portion of the sac. In treatment of such an aneurysm, the braided mesh loop of the present invention is caused to expand upon unsheathing as it is pushed from the distal end of the micro delivery catheter.

The bifurcated vessels extending from the main vessel are typically not occluded by the expanded mesh loop of the scaffold. The bifurcated vessels do however, serve to limit the continued free expansion of the wire mesh loop, resulting in a generally horseshoe shape (or may be characterized as a “flame” shape with a transition to a sphere shape) of the wire mesh loop by which the aneurysm is being treated.

A further embodiment for the deployment of a wire mesh loop adjacent the neck of an aneurysm is contemplated by the maintenance of a proximal portion of the braided wire mesh loop remaining within the inside distal end of the micro delivery catheter. In such an orientation, the wire mesh loop does not provide the open central portion along the inner periphery of the loop as in the aforementioned previous embodiment thereof. Blood flow however, may extend around the outer periphery of the wire mesh loop between the first and second side vessels and from the main vessel channel. Such partial deployment of the mesh loop out of the distal end of the delivery catheter permits control of the molding of the mesh loop to the existing target environment and also alter the flow of blood in the local environment.

Once the wire mesh loop has been expanded distal of the micro delivery catheter and its distalmost bulbous end thereof is pressed against the opening of the neck of the aneurysm, a further micro delivery catheter may be presented through the main vessel and through the opening in the central portion of the loop and into the aneurysm itself, so as to deliver embolitics such as, for example coils, wires, and/or liquid embolic material.

Such a further micro delivery catheter may also be presented alongside the primary micro delivery catheter which further micro delivery catheter may be delivered through the wall of the wire mesh braid and into the aneurysm itself for a similar delivery of embolic material.

A further embodiment of the wire mesh loop arrangement contemplates the dis-attachment of the loop after it has been discharged from the distal end of the micro catheter delivery arrangement. Such a disattached loop may be pushed into place by a push shaft arranged through the micro delivery catheter. The push shaft may have in one embodiment a recess on its distalmost end, for pushing upon the proximal end of the juncture or termination point of the wire mesh loop.

A yet further embodiment of the present invention is contemplated wherein the wire mesh loop is attached to the distalmost end of a tubular control rod which itself is longitudinally displaced through the micro catheter delivery arrangement. Use of such a tubular or hollow control rod attached to the proximal end of the braided mesh loop permits embolic material to pass through the lumen of the control tube and into the aneurysm. Such embolic material may be introduced into the aneurysm by a further hollow tubular member longitudinally displaceable out of the distal end of the micro delivery catheter.

The woven wire mesh loop in yet a further embodiment, contemplates a change in “pic” count along its length, so that the distalmost portion of the wire mesh loop may have a denser configuration of wire mesh thereat. Such a denser portion of wire mesh would assist in the occlusion of the aneurysm itself upon the delivery of embolic material therewithin. In a further embodiment of the present invention, the mesh loop may be coated with a fabric or polymer on all or a portion of the mesh scaffold. Such a coating may be lased in certain areas to permit blood to perfuse those selected areas of the coating.

A yet further contemplation of the methodology of treating an aneurysm at a bifurcation includes the dis-attachment of a wire mesh loop directly into the sac of an aneurysm itself, and withdrawing the micro delivery catheter therefrom. The expanded wire mesh loop will fill the aneurysm sac thus avoiding necessity of further embolic material being placed therein. Delivery of such a wire mesh loop into the aneurysm sac would be accomplished by the micro delivery catheter being directed into the sac itself and the wire mesh loop being discharged therewithin. Drug infusion into the aneurysm, in a further embodiment is arranged by porous hypotubes in their distal ends for delivery of drugs to a treatment site.

A yet further embodiment of the present invention contemplates a wire mesh loop having its proximal ends thereof each respectively controlled by a push wire attached thereto. Enlargement of the wire mesh loop would be effected by relative motion of one or the other of those two push wires with respect to the distal end of the micro delivery catheter. The relative diameter might be changed by pulling back on one wire (or both push wires) while holding the other wire in place.

The invention thus comprises an aneurysm buttressing arrangement for covering an aneurysm opening in an intracranial aneurysm, for temporary placement thereadjacent, to prevent escape of embolitic agents from that aneurysm. The arrangement comprises an elongated delivery wire having a proximal end and a distal end. A scaffold of expandable wires is arranged on the distal end of the delivery wire, the scaffold comprising a generally “U” shaped arrangement of wire mesh thereattached. The scaffold of wires has a bulbous distal end and a pair of narrower proximal ends. The scaffold may be of circular cross-section. The bulbous end may have a higher density of wires arranged thereat than the wire density of the wires at the proximal ends of the scaffold. The scaffold of wires or shaped inflated balloon in further embodiments has a central opening. The central opening in the scaffold of wires or balloon is variable in size. The scaffold may be dis-attachable from the delivery wire. The scaffold may be delivered to a target site by a microcatheter having a distal end. The delivery wire may be hollow. A further micro delivery catheter may be movably arranged in the hollow delivery wire. The further micro delivery wire may be arranged to supply embolics to the aneurysm. The aneurysm may be disposed on a bifurcated blood vessel for engaging the scaffold. The delivery wire may have a recess on its distal end for control of a scaffold thereat.

The invention may also include a method of buttressing an intracranial aneurysm in a wall of a vessel comprising one or more of the steps of: transluminally positioning a compressed, generally “U” shaped scaffold of wires at a distal end of a delivery catheter; introducing the distal end of the delivery catheter adjacent a neck of an aneurysm in the wall of the vessel; moving the scaffold of wires distally out of the delivery catheter and against the neck of the aneurysm; and expanding the scaffold into a generally “U” shaped mesh of wires. The method may include: forming a central opening in the generally “U” shaped mesh of wires; flowing blood in the vessel through the central opening of the mesh to prevent injury to a patient with the aneurysm; introducing a further delivery catheter into the vessel for introduction of embolitic material into the aneurysm; attaching a proximal end of the scaffold to a distal end of a delivery wire movably disposed in the delivery catheter; dis-attaching the scaffold from the delivery wire upon introduction of the scaffold against the aneurysm; attaching a pair of proximal ends of the scaffold to a distal end of a pair of delivery wires movably disposed in the delivery catheter.

The invention may also include a method of buttressing an intracranial aneurysm in a wall of a vessel comprising one or more of the steps of: placing a scaffold adjacent the aneurysm; creating a central opening in the scaffold; directing blood through the central opening in the scaffold; and introducing a embolic material through the opening and into the aneurysm; moving the scaffold into a delivery catheter; displacing the scaffold from the delivery catheter; expanding the scaffold from a compressed orientation into an expanded U-shaped configuration into a buttressing arrangement against the aneurysm, the scaffold having a bulbous distal end and a pair of narrower proximal ends; attaching the proximal ends of the scaffold to a distal end of a control wire; moving the control wire to effect size and shape of the scaffold.

### BRIEF DESCRIPTION OF THE DRAWINGS

The objects and advantages of the present invention will become more apparent when viewed in conjunction with the following drawings, in which:

FIG. 1 is a side elevational view, partly in section of a wire mesh loop extending from the distalmost end of a micro delivery catheter;

FIG. 2 is a view of a wire mesh loop extending only partially from the distalmost end of a micro delivery catheter;

FIG. 3 is a side elevational view, in section, of a wire mesh loop collapsed in the distalmost end of a micro delivery catheter;

FIG. 4 is a side elevational view of an aneurysm at a bifurcated vessel, with a wire mesh loop shown having been delivered from a micro delivery catheter;

FIG. 5 is a view similar to FIG. 4 showing the wire mesh loop partially retained within the distalmost end of the micro dilivery catheter at the site of the aneurysm;

FIG. 6 is a side elevational view, in section, showing a bifurcated vessel with an aneurysm and a wire mesh loop distally delivered from a micro delivery catheter, with a further micro catheter arranged through an opening in the loop and into the aneurysm at the bifurcated vessel;

FIG. 7 is a view similar to FIG. 6, showing a further micro delivery catheter extending through a side wall of the extended wire mesh loop adjacent the aneurysm;

FIG. 8 is a side elevational view, partially in section, showing a micro delivery catheter and push shaft being removed from a dis-attached wire mesh loop placed adjacent the neck of an aneurysm at a bifurcated vessel;

FIG. 8A is a side elevational view, in section, showing a push shaft adapted to push a dis-attached loop wire mesh into an aneurysm;

FIG. 9 is a side elevational view of a wire mesh loop attached to a hollow push rod;

FIG. 10 is a side elevational view of a hollow microdelivery catheter with a braided wire mesh loop extended therefrom with a control rod for delivering embolitic agents, extending beyond the distal end of the loop;

FIG. 11 is a view, partially in section showing a wire mesh loop disattached from a delivery catheter, with an occlusive braid at its distalmost end;

FIG. 12 is a side elevational view of portion of a braided wire mesh loop showing variations in the braid count along certain portions of that loop;

FIG. 12A is a side view of an expanded wire mesh loop showing a higher braid count along the distalmost contours of the wire mesh loop;

FIG. 13 is a side elevational view of a wire mesh loop placed into the aneurysm sac at a bifurcated vessel with the microdelivery catheter being withdrawn therefrom;

FIG. 14 is a side elevational view of a microdelivery catheter entering into the aneurysm sac with a wire mesh loop about to be delivered therewithin;

FIG. 15 is a side elevational view, partly in section, of a wire mesh loop being delivered and dimensionally controlled by a pair of displaceable delivery and control wires;

FIG. 16 is a side elevational view of a control rod within a delivery catheter, showing a wire mesh loop wrapped about the distal end of that rod for delivery to a target site;

FIG. 17 shows a side elevational view of the wire mesh loop being inserted into a delivery catheter;

FIG. 18 shows a wire mesh loop in an apex of a bifurcated aneurysm with vessel branches extending adjacent a mid portion of the mesh loop; and

FIG. 19 the wire mesh loop of FIG. 18 and vessel arranged from ninety degrees perspective about its vertical axis.

### DETAILED DESCRIPTION OF THE PREFERRED EMBODIMENTS

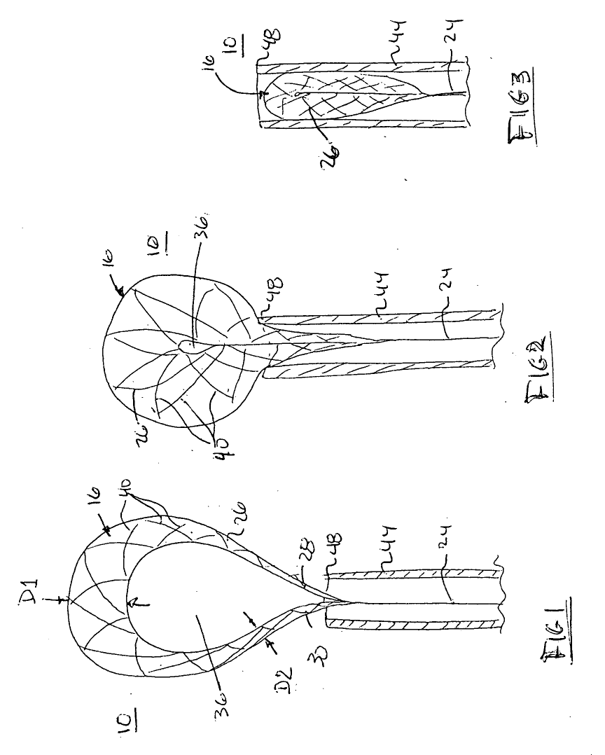

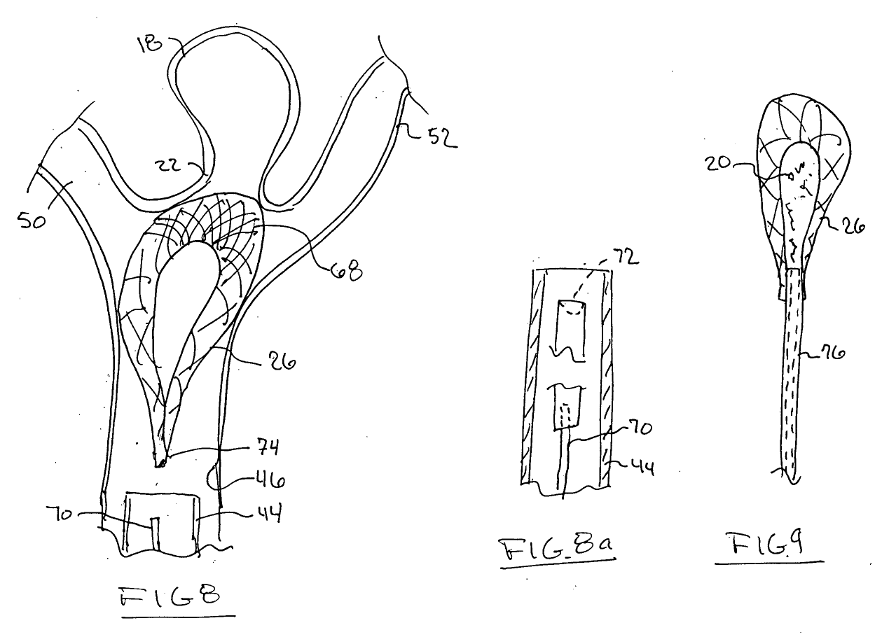

Referring now to the drawings in detail, and particularly to FIG. 1, there is shown the present invention which comprises a bifurcated aneurysm treatment apparatus 10 as represented in FIGS. 1, 2 and 3 including a method of filling and buttressing an intracranial aneurysm 12, as represented in FIG. 4 et seq., such as may be located at the juncture of a bifurcated vessel arrangement 14, shown in FIGS. 4, 5, and 6 et seq. The method includes the steps of transluminally positioning a buttress scaffold 16 across or in an opening of an aneurysm 12 at the location of a bifurcated vessel 14 so as to block off and isolate that aneurysm cavity (or sac) 18 in a sidewall of that bifurcated vessel 14, as represented in FIGS. 4, 5, 6, 7 and 8. Media such as embolitic agents, coils, and/or polymers 20 may then be introduced into that cavity within the sidewall of the bifurcated vessel 14, as represented in FIGS. 6 and 7. The aneurysm or cavity 12 is often of a bulbous shape having a neck portion 22 no greater than about one-half of the diameter of the bulbous portion.

The bulbous scaffold 16 is arranged on the distal end of an elongated delivery wire or push wire 24 much like a guide wire, and is shown for example in FIGS. 1, 2, 3, 4, 5, 6 and 7. The scaffold 16 itself may be preferably comprised in one embodiment of a balloon or in another preferred embodiment which will be described in detail, of a mesh or braid of wire, comprised of a memory metal or polymeric fibers and/or a plastic, or a coweave combination thereof shaped generally as a prolate spheroid and disposed in the target vessel in the manner of a generally U-shaped loop 26 as best represented in FIGS. 1, 4, 6, 7, 8, 9, 10, 12a and 15. The loop 26 is defined by two proximal ends 28 and 30 which taper into a thinner diameter D2 from a larger diameter D1 at the distalmost end of the loop 26, as represented in FIG. 1. The loop 26, both at its distalmost end and at its proximal most ends are preferably circular in cross section, but may be oval or somewhat rectangular. The loop 26 is expandable and defines a variable central opening 36 along its innermost periphery thereof, as represented in FIGS. 1 and 4.

The buttress scaffold 16 may be comprised in one embodiment for example, of an thin, inflatable balloon as aforesaid, or in another preferred embodiment for example, of an array of flexible interwoven nitinol wires, stainless steel wires or filaments of polymer 40. A typical pic per inch count for the braided mesh ranges from a low of about 1 to 20 ppi to a high of about 1000 ppi based on the radial expansion required for a given target vessel aneurysm. Target vessel sizes ranged from about 1 or 2 mm to about 6 mm. The individual carrier wire 40 utilized in a braided mesh loop 26 may have a diameter ranging from about 0.00075 inch to about 0.003 inches. The carrier-counts for the braided mesh may preferably range for example, from about 4 to about 128 carriers, with the denser braid configurations designed to permit enhanced resistance to embolic material leaking beyond the aneurysm neck 22 into the parent vessel while the scaffold loop 16 is in place. Typical braided mesh construction is either a one over, one under, or a two over, two under, filament crossing pattern, as represented in FIG. 12. The braided mesh loop 26 may be formed by heat setting, mechanical bending or other means so as to take on a set expanded shape. The braided mesh loop 26 may also be electrically polished, and/or chemically etched along a partial section or its entirety. Surface coatings to increase the fluoroscopic visibility of the scaffold device or at distinct marker points along the loop may also be applied. Hydrophilic coatings, friction reducing coatings or any coating or medicament which may speed healing or enhance the surface properties of an operation associated with the insertion of such a braided mesh loop 26 may be utilized therewith.

The control shaft in one preferred embodiment may be solid such as a stainless steel wire sized appropriately to the overall device size. Such a control shaft may be Teflon® coated either partially or along its entire length. The braided loop of the present invention is intended to act as an atraumatic means of supporting the scaffold device against the main vessel branch of the bifurcation.

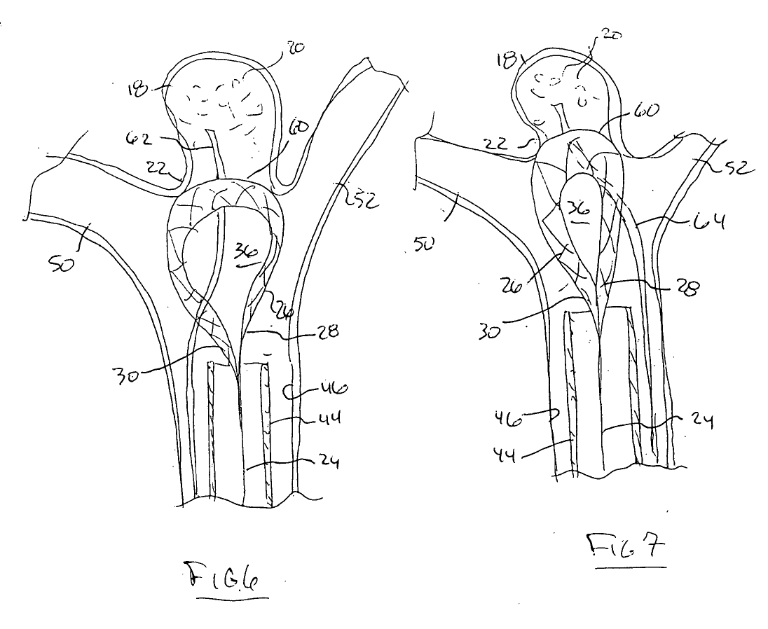

The wire mesh loop 26 is preferably delivered through a micro delivery catheter 44 pushed through the main vessel 46 and into the bifurcated target area, 18 as represented in FIGS. 4, 5, 6, 7 and 8. The push wire 24 proximal of the mesh loop 26 may be advanced so as to only extend the wire mesh loop only a portion of the way out of the distal end 48 of the micro delivery catheter 44, as represented in FIGS. 2 and 5. This will permit one means of control for the ultimate diameter of the opened distalmost wire mesh loop 26.

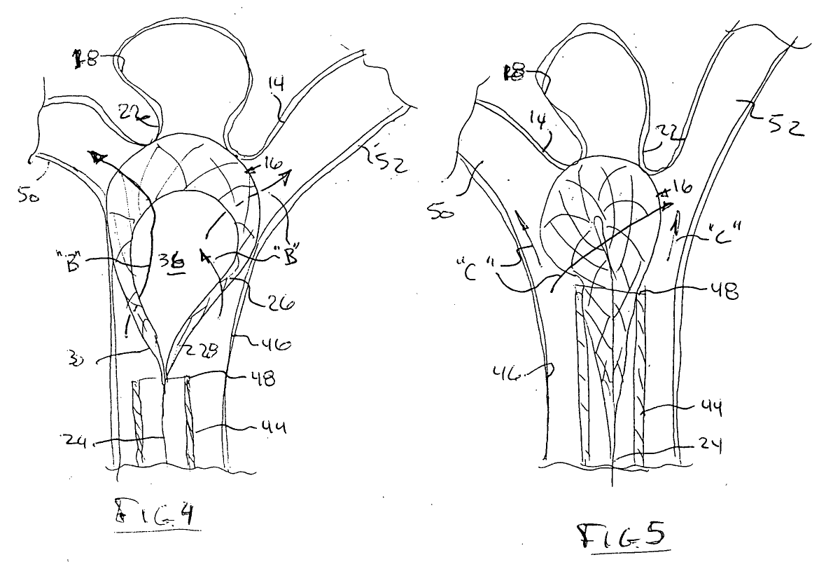

The wire mesh loop 26 in its “delivery” configuration in a distal end of a micro delivery catheter is enclosed in a fully collapsed orientation, as shown in FIG. 3. The inside diameter of a typical micro delivery catheter 44 in which such a wire mesh loop 26 would be loaded and tracked through the vasculature of a patient may range in diameter from about 0.010 inches to about 0.027 inches. The wire mesh loop 26 is advanced to become a target anatomy conforming scaffold 16 and may be eventually removed from the aneurysm site by being withdrawn through such a micro delivery catheter 44. The collapse of a loop of braided mesh 26 upon retrieval and/or removal of such a wire mesh loop device is a result of the braided mesh loop 26 passing over and being compressed by the annular distalmost lip 48 of the micro delivery catheter 44.

A typical aneurysm “sac” 18 is often located at the locus of intersection of a first and a second blood vessel or channel 50 and 52 which may be fed by a main vessel channel 46. The aneurysm sac 18 at such a bifurcation typically has its neck portion 22 approximately one-half of the diameter of the enlarged or bulbous portion of the sac 18. In treatment of such an aneurysm, the braided mesh loop 26 of the present invention is caused to expand upon unsheathing as it is pushed from the distal end of the micro delivery catheter 44, as represented in FIG. 3, and into engagement with the neck 22 to become the scaffold 16 as represented for example, in FIGS. 4, 5, 6 and 7.

The bifurcated vessels 50 and 52 extending from the main vessel 46 are typically not occluded by the expanded mesh loop of the scaffold 16, the blood flow passing through the central opening 36 of the loop 26 and around the ends 28 and 30, as represented by arrows “B” in FIG. 4. The bifurcated vessels do however, serve to limit the continued free expansion of the wire mesh loop 26, resulting in a generally horseshoe shape of the wire mesh loop by which the aneurysm is being treated.

A further embodiment for the deployment of a wire mesh loop 26 adjacent the neck 22 of an aneurysm 18 is contemplated by the maintenance of a proximal portion of the braided wire mesh loop 26 remaining within the inside distal end of the micro delivery catheter 44, as shown in FIGS. 2 and 5. In such an orientation, the wire mesh loop 26 does not provide an enlarged open central portion 36 along the inner periphery of the loop as in the aforementioned previous embodiment thereof. Blood flow however, may extend around the outer periphery of the wire mesh loop between the first and second side vessels 50 and 52, and from the main vessel channel 46, as represented by the arrows C in FIG. 5.

An example of a construction of such a mesh scaffold of the present invention may be as follows: cut approximately 2″ of Duke Braid #36 arranged on a mandrel; fully expand about 1.5 cm. of the braid on the mandrel along a midpoint thereof; constrain the braid in its fully expanded position by wrapping the ends of the braid to the mandrel OD using a length of 304 SS wire 0.004″ which will withstand high temperatures; heat the expanded portion to 1100 degrees F. for 5-10 minutes, quenching immediately in water after removal of the braid from the heat; remove the SS wires: pull on ends of heat set braid to desired OD of scaffold device and place over heat source to set in position, and quench; trim ends of braid with one end of non-expanded braid about 7-10 mm longer than the other end; slide the end of an 8″ piece of PTFC heat sink (expanded 0.019 recovered 0.005, 0.0016 wall) over the ends of the heat set braid; heat only the distalmost 3 mm of heat sink at 700 degrees F. to pull down on the braid; and slide Duke Empirical push wire, drawing #SD-1053 into open end of heat sink tube and push up until it contacts heat shrunk region and is aligned next to the long and short expanded braid legs and heat shrink the entire 8″ length of tube down onto braid and push wire.

Once the wire mesh loop 26 has been expanded distal of the micro delivery catheter 44 and its distalmost bulbous end 60 thereof is pressed against the opening of the neck 22 of the aneurysm 18, a further micro delivery catheter 62 may be presented through the main vessel 46 and through the opening 36 in the central portion of the loop 26 and into the aneurysm 18 itself, so as to deliver embolics 20 such as, for example coils, wires, and/or liquid embolic material, as represented in FIG. 6.

Such a further micro delivery catheter 64 may also be presented alongside the primary micro delivery catheter 44 which further micro delivery catheter 64 may be delivered through the wall of the wire mesh braid loop 26, as represented in FIG. 7, and into the aneurysm 18 itself for a similar delivery of embolic material 20.

A further embodiment of the wire mesh loop arrangement contemplates the dis-attachment of a mesh loop 68 from a push wire 70 to become a scaffold 16 after it has been discharged from the distal end of the micro delivery catheter 44 arrangement is represented in FIG. 8. Such a dis-attached loop 68 may be pushed into place by the push shaft 70 movably arranged through the micro delivery catheter 44. The push wire 70 may have a recess 72 in a sleeve 73 on its distalmost end, as represented in FIG. 8a, for pushing upon the proximal end 74 of the juncture or termination point of the dis-attached wire mesh loop 68.

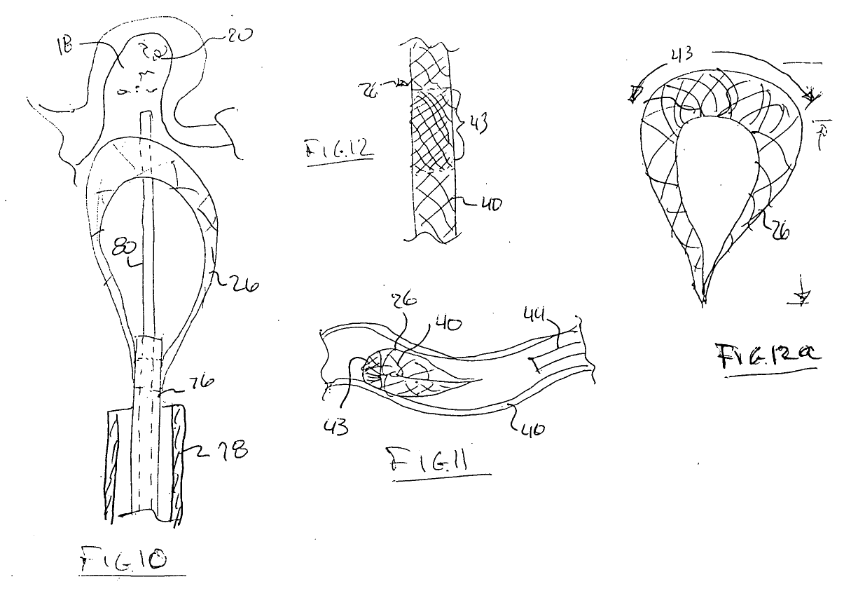

A yet further embodiment of the present invention, shown in FIG. 9, is contemplated wherein the wire mesh loop 26 is attached to the distalmost end of a tubular control rod 76, which rod 76 itself may also be longitudinally displaced through a micro catheter delivery arrangement 78, as shown in FIG. 10. Use of such a tubular or hollow control rod 76 attached to the proximal end of the braided mesh loop 26 permits embolic material 20 to pass through the lumen of the control tube 76 and into the aneurysm 18. Such embolic material 20 may also be introduced into the aneurysm by a further hollow tubular member 80 longitudinally displaceable out of the distal end of the rod 76 and through the micro delivery catheter 78, as represented in FIG. 10.

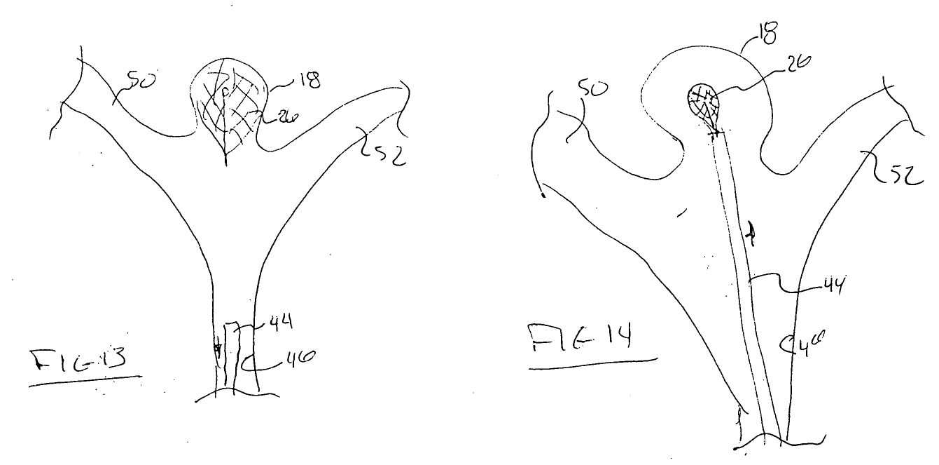

The woven wire mesh loop 26 in yet a further embodiment, contemplates a change in “pic” count of wire 40 along its length, so that the distalmost portion of the wire mesh loop 26 may have a denser configuration 43 of wire mesh 40 at a central portion, as represented in FIGS. 11, 12 and 12a. Such a denser portion 43 of wire mesh 40 would assist in the occlusion of the aneurysm itself upon the delivery of embolic material therewithin while permitting blood to pass through a portion of the wall or through a central opening of the scaffold.

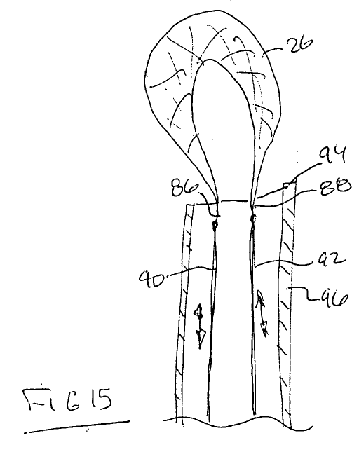

A yet further contemplation of the methodology of treating an aneurysm 18 at a bifurcation includes the dis-attachment of a wire mesh loop 26 directly into the sac of an aneurysm itself, and withdrawing the micro delivery catheter 44 therefrom, as represented in FIG. 13. The expanded wire mesh loop 26 will fill the aneurysm sac 18 thus avoiding necessity of further embolic material being placed therein. Delivery of such a wire mesh loop 26 at least partially into the aneurysm sac 18 would be accomplished by the micro delivery catheter 44 being directed into the sac 18 itself, as represented in FIG. 14, and then the wire mesh loop 26 being discharged and dis-attached by electric current or mechanical release means therewith, as shown in FIG. 13.

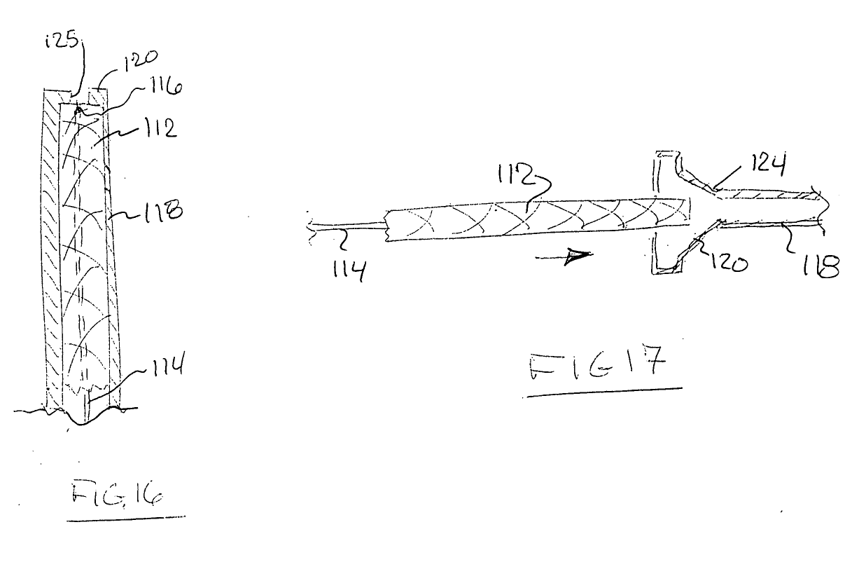

A yet further embodiment of the present invention shown in FIG. 15 comprises a wire mesh loop 26 having its proximal ends 86 and 88 thereof each respectively controlled by a pair of push wires 90 and 92 attached thereto. Enlargement or diminution of the wire mesh loop 26 would be effected by relative motion of one or the other (or both) of those two push wires 90 and 92 with respect to the distal end 94 of the micro delivery catheter 96. The relative diameter might be changed by pulling back on one wire 90 (or both push wires) while holding the other wire 92 in place.

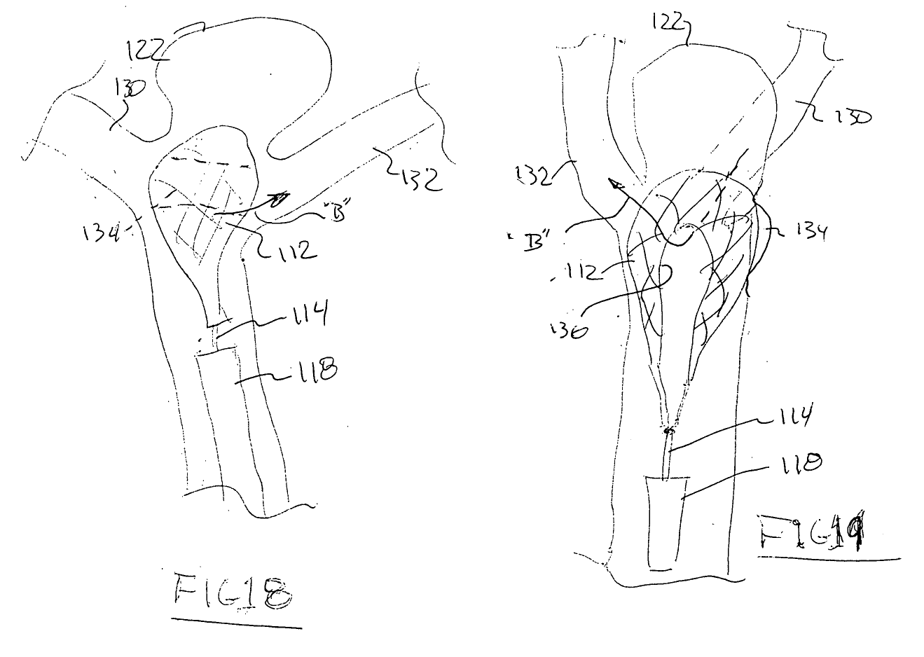

FIG. 16 discloses a wire mesh loop or scaffold 112 wrapped about the distalmost portion of a control or delivery wire 114. The mesh loop 112 is attached to the delivery wire 114 at its distalmost end 116. The mesh loop 112 is disposed in the distalmost end of a micro delivery catheter 118. The delivery catheter 118 has a radially inwardly directed lip 120 surrounding a deployment opening 125. The delivery wire 114 is utilized for deploying the mesh loop 112 into the vessel bifurcation 122, as represented in FIGS. 18 and 19. Distal pushing of the control wire 114 relative to the catheter 118 pushes the mesh loop 112 out of the end of the catheter 118. The mesh loop 112 is loaded into the delivery catheter 118 by insertion into a flared opening 120 at its proximal end 124, as represented in FIG. 17. The bifurcation 122 shown in FIG. 19 represents the bifurcation shown in FIG. 18, rotated about a vertical axis by 90 degrees. This attempts to show how a first vessel 130 may be in communication with a second branch vessel 132 by a connecting channel 134. The mesh loop 112 having a central opening 136 to also permit flow of blood therethrough between the various vessels 130 and 132, as indicated by arrow “B” in FIGS. 18 and 19.

We claim:

1. An aneurysm buttressing arrangement for covering an aneurysm opening in an intracranial aneurysm, for temporary placement thereadjacent, to prevent escape of embolitic agents from that aneurysm, said arrangement comprising:
an elongated delivery wire having a proximal end and a distal end;
an expandable scaffold arranged on said distal end of said delivery wire, said scaffold comprising a generally “U” shaped expandable arrangement of fluid permeable material secured to said delivery wire.

2. The aneurysm buttressing arrangement as recited in claim 1, wherein said expandable scaffold has a bulbous distal end and a pair of narrower proximal ends which narrower proximal ends are attached to said delivery wire.

3. The aneurysm buttressing arrangement as recited in claim 1, wherein said scaffold is of comprised of a wire mesh having a circular cross-section.

4. The aneurysm buttressing arrangement as recited in claim 3, wherein said bulbous end has a higher density of wires arranged thereat than the wire density of said wires at said proximal ends of said scaffold.

5. The aneurysm buttressing arrangement as recited in claim 3, wherein said fluid permeable scaffold of wires has a central opening.

6. The aneurysm buttressing arrangement as recited in claim 5, wherein said central opening in said scaffold of wires is variable in size.

7. The aneurysm buttressing arrangement as recited in claim 1, wherein said scaffold is dis-attachable from said delivery wire.

8. The aneurysm buttressing arrangement as recited in claim 1, wherein said scaffold is delivered to a target site by a micro catheter having a distal end.

9. The aneurysm buttressing arrangement as recited in claim 8, wherein said delivery wire is hollow.

10. The aneurysm buttressing arrangement as recited in claim 9, wherein a further micro delivery catheter is movably arranged in said hollow delivery wire.

11. The aneurysm buttressing arrangement as recited in claim 10, wherein said further micro delivery wire is arranged to supply embolitics to said aneurysm.

12. The aneurysm buttressing arrangement as recited in claim 1, wherein said aneurysm is disposed on a bifurcated blood vessel for engaging said scaffold.

13. The aneurysm buttressing arrangement as recited in claim 1, wherein said delivery wire has a recess on its distal end for control of a scaffold thereat.

14. A method of buttressing an intracranial aneurysm in a wall of a vessel comprising:
transluminally positioning a compressed, generally “U” shaped expandable scaffold at a distal end of a delivery catheter;
introducing said distal end of said delivery catheter adjacent a neck of an aneurysm in said wall of said vessel;
moving said expandable scaffold distally out of said delivery catheter and against said neck of said aneurysm; and
expanding said scaffold into a generally flexible, fluid passable “U”-shaped member.

15. The method as recited in claim 14, including:
forming a central opening in said generally “U”-shaped scaffold.

16. The method as recited in claim 15, including:
flowing blood in said vessel through said central opening of said scaffold to prevent injury to a patient with said aneurysm.

17. The method as recited in claim 14, including:
introducing a further delivery catheter into said vessel for introduction of embolitic material into said aneurysm.

18. The method as recited in claim 14, including:
attaching a proximal end of said scaffold to a distal end of a delivery wire movably disposed in said delivery catheter.

19. The method as recited in claim 18, including:
dis-attaching said scaffold from said delivery wire upon introduction of said scaffold against said aneurysm.

20. The method as recited in claim 14, including:
attaching a pair of proximal ends of said scaffold to a distal end of a pair of delivery wires movably disposed in said delivery catheter.

21. The method as recited in claim 14, wherein said scaffold comprises a mesh of flexible target conformable wires.

22. A method of buttressing an intracranial aneurysm in a wall of a vessel comprising:
placing a scaffold adjacent said aneurysm;
creating a central looped opening in said scaffold;
directing blood through said central opening in said scaffold; and
introducing a embolic material through said opening and into said aneurysm.

23. The method as recited in claim 22, including:
moving said scaffold into a delivery catheter;
displacing said scaffold from said delivery catheter;
expanding said scaffold from a compressed orientation into an expanded U-shaped configuration into a buttressing arrangement against said aneurysm, said scaffold having a bulbous distal end and a pair of narrower proximal ends.

24. The method as recited in claim 23, including:
attaching said proximal ends of said scaffold to a distal end of a delivery wire.

25. The method as recited in claim 23, including:
moving said delivery wire relative to said delivery catheter to effect size and shape of said scaffold.

26. The method as recited in claim 23, including:
wrapping said scaffold about the distal end of said delivery wire to permit snug insertion of said scaffold into said delivery catheter.

27. The method as recited in claim 26, including:
arranging a radially inwardly directed lip on a distalmost end of said delivery catheter; and
pushing said scaffold into a flared opening at a proximal end of said delivery catheter so as to load said delivery catheter with said scafold.

28. The method as recited in claim 23, including:
fabricating said scaffold of a plurality of wound wires to effect a generally tubular loop of wire mesh; and
weaving a higher density of wound wires on a distal end portion of said scaffold and a lower density of wound wires on a proximal end of said scaffold, said proximal end of said scaffold comprising a joining of each end of said wire mesh to form said loop.

29. The method as recited in claim 23, including:
attaching a delivery wire on each end of said wire mesh on said proximal end of said scaffold to increase control of said scaffold.

30. The method as recited in claim 29, including:
moving one of said delivery wires with respect to another of said delivery wires to effect a deployed shape of said wire mesh relative to said aneurysm.

31. The method as recited in claim 23, including:
separating said scaffold from said delivery wire after deployment of said scaffold at said aneurysm.

32. The method as recited in claim 23, wherein said scaffold has at least a partial coating of film thereon.

33. The method as recited in claim 23, wherein said scaffold is comprised of a balloon.
